(19) United States
(12) Patent Application Publication
(10) Pub. No.: US 2014/0342025 A1
(43) Pub. Date: Nov. 20, 2014

(54) ARJUNOLIC ACID FOR INCREASING SEBUM PRODUCTION

(71) Applicant: EVONIK INDUSTRIES AG, Essen (DE)

(72) Inventors: Mike Farwick, Essen (DE); Benjamin Tuchscherer, Dortmund (DE); Matthias Mentel, Dortmund (DE)

(73) Assignee: EVONIK INDUSTRIES AG, Essen (DE)

(21) Appl. No.: 14/355,232

(22) PCT Filed: Oct. 5, 2012
(86) PCT No.: PCT/EP2012/069703

§ 371 Date: Apr. 30, 2014

(30) Foreign Application Priority Data
Oct. 31, 2011 (DE) 102011085493.2

(51) Int. Cl. A61K 8/36; A61K 8/34; A61Q 19/00; A61K 8/97

(52) U.S. Cl. CPC A61K 8/36; A61K 8/97; A61K 8/345; A61Q 19/007
USPC 424/769; 514/557

(57) ABSTRACT

The invention relates to the use of arjunolic acid for increasing sebum production, and to preparations comprising arjunolic acid and 1,2-pentanediol.

### FIELD OF THE INVENTION

The invention relates to the use of arjunolic acid for increasing sebum production and to compositions comprising arjunolic acid and 1,2-pentanediol.

### PRIOR ART

Postmenopausal women naturally have reduced sebum production of the skin and consequently have a tendency towards dry skin.

An inadequate approach to combating this natural ageing phenomenon lies in the administration of isoflavones, which have phytohormone effect and are said to substitute the postmenopausally lowered oestrogen level. Isoflavones have the disadvantage that their effectiveness is limited, and they are difficult to formulate.

It was an object of the invention to provide an active ingredient which can increase sebum production in human skin, in particular in postmenopausal women.

### DESCRIPTION OF THE INVENTION Surprisingly, it has been found that arjunolic acid is able to achieve the object underlying the invention.

The subject matter of the present invention is therefore the use of arjunolic acid for increasing sebum production.

A further subject matter of the invention is a composition comprising arjunolic acid and 1,2-pentanediol.

A yet further subject matter of the invention is a process for preparing a formulation starting from the aforementioned composition.

It is advantageous that arjunolic acid exerts further advantageous properties upon topical application.

It is another advantage that the composition according to the invention permits a simple formulation of arjunolic acid.

It is another advantage that the composition permits improved bioavailability of arjunolic acid in cosmetics.

Unless stated otherwise, all of the stated percentages (%) are percentages by mass.

The subject matter of the present invention is arjunolic acid for increasing sebum production, and the cosmetic use of a formulation comprising arjunolic acid for increasing sebum production.

These two subjects of the present invention preferably find application on skin of postmenopausal women.

For these two subjects, it is equally advantageous if asiatic acid is additionally present, preferably in a weight ratio of arjunolic acid to asiatic acid of 1:1 to 20:1, in particular from 2:1 to 15:1.

On account of the good availability, it would seem preferable for these two subjects of the present invention to use an extract of Terminalia arjuna, in particular terminalia arjuna bark extract.

For these two subjects of the present invention, it has proven to be advantageous if 1,2-pentanediol is additionally present, preferably in a weight ratio of arjunolic acid to 1,2-pentanediol of 1:500 to 1:4, in particular from 1:19 to 1:9.

Since arjunolic acid is difficult to incorporate into stable formulations on account of its poor solubility in pharmaceutical and cosmetic solvents, a composition comprising

0.2% by weight to 20% by weight, preferably 2% by weight to 12% by weight, in particular 5% by weight to 10% by weight, of arjunolic acid and

80% by weight to 99.8% by weight, preferably 88% by weight to 98% by weight, in particular 90% by weight to 95% by weight, of 1,2-pentanediol,

where the percentages by weight refer to the total composition, makes a contribution to the object underlying the invention.

Surprisingly, 1,2-pentanediol is a good solvent for arjunolic acid and the composition according to the invention permits a problem-free formulation of arjunolic acid in pharmaceutical and/or cosmetic formulations.

A composition preferred according to the invention is characterized in that it additionally comprises asiatic acid, preferably in a weight ratio of arjunolic acid to asiatic acid of 1:1 to 20:1, in particular from 2:1 to 15:1.

Here too, on account of the good availability, it would seem preferable to use an extract of Terminalia arjuna, in particular terminalia arjuna bark extract.

Moreover, it has been found that the bioavailability of arjunolic acid is drastically increased for those pharmaceutical and/or cosmetic formulations which have been prepared using the composition according to the invention.

Consequently, a further subject matter of the present invention is a process for preparing a formulation, in particular a cosmetic or pharmaceutical formulation, comprising the process steps

A) provision of a composition according to the invention,

B) blending of the provided composition with further components of the formulation.

Suitable substances used as further components in process step B) are, for example, emollients, emulsifiers and surfactants, thickeners, viscosity regulators, stabilizers, UV photoprotective filters, antioxidants, hydrotropes (or polyols), solids and fillers, film formers, pearlescent additives, deodorant and antiperspirant active ingredients, insect repellents, self-tanning agents, preservatives, conditioners, perfumes, dyes, biogenic active ingredients, care additives, superfatting agents and further solvents such as, for example, water.

In particular, at least one further biogenic active ingredient is used as further components in process step B); biogenic active ingredients are to be understood as meaning, for example, tocopherol, tocopherol acetate, tocopherol palmitate, ascorbic acid, polyphenols, deoxyribonucleic acid, coenzyme Q10, retinol, AHA acids, amino acids, hyaluronic acid, alpha-hydroxy acids, isoflavones, polyglutamic acid, creatine (and creatine derivatives), guanidine (and guanidine derivatives), pseudoceramides, essential oils, peptides, protein hydrolysates, plant extracts, bisabolol, allantoin, panthenol, phytantriol, idebenone, liquorice extract, glycyrrhizidine and idebenone, scleroglucan, fl-glucan, santalbic acid and vitamin complexes. Examples of plant extracts are horse chestnut extract, chamomile extract, cucumber extract, rosemary extract, black and red currant extract, birch extract, rosehip extract, algae extract, green tea extract, aloe extract, ginseng extract, gingko extract, grapefruit extract, calendula extract, camphor, mangosteen extract, cystus extract, oat extract, oregano extract, raspberry extract, strawberry extract, etc. The biogenic active ingredients can also include the so-called barrier lipids, examples being ceramides, phytosphingosine and derivatives, sphingosine and derivatives, sphinganine and derivatives, pseudoceramides, phospholipids, lysophospholipids, cholesterol and derivatives, cholesteryl ester, free fatty acids, lanolin and derivatives, squalane, squalene and related substances. Within the context of the invention, the biogenic active ingredients also include antiacne ones, such as e.g. benzoyl peroxide, phytosphingosine and derivatives, niacinamide hydroxybenzoate, nicotinaldehyde, retinoic acid and derivatives, salicylic acid and derivatives, citronellic acid etc. and anti-cellulite ones such as e.g. xanthine compounds such as caffeine, theophylline, theobromine and aminophylline, carnitine, carnosine, salicyloyl phytosphingosine, phytosphingosines, santalbic acid etc., as well as antidandruff agents such as, for example, salicylic acid and derivatives, zinc pyrithione, selenium sulphide, sulphur, ciclopiroxolamine, bifonazole, climbazole, octopirox and actirox etc., as well as astringents such as e.g. alcohol, aluminium derivatives, gallic acid, pyridoxine salicylate, zinc salts such as e.g. zinc sulphate, acetate, chloride, lactate, zirconium chlorohydrates etc. Bleaches such as kojic acid, arbutin, vitamin C and derivatives, hydroquinone, turmeric oil, creatinine, sphingolipids, niacinamide, etc. can likewise be included among the biogenic active ingredients.

In a further embodiment, the further components used in process step B) are partition modifiers, such as, for example, benzyl alcohol, alpha-bisabolol, cetyl alcohol, chitosan, decanol, decyl methyl sulphoxide, diethylene glycol monoethyl ether, dimethylformamide, dimethylacetamide, dimethyl sulphoxide, EDTA, ethylene glycol, 2-pyrrolidone, N-methyl-2-pyrrolidone, 1-dodecylazacycloheptan-2-one, 4-decyloxazolidin-2-one, ethanol, glycerol, lauric acid, lauryl alcohol, lecithin, urea, oleic acid, linoleic acid, linolenic acid, dimethyl isosorbide, isopropyl myristate, propylene glycol, sodium lauryl sulphate, cetyltrimethylammonium bromide, dodecylbetaine, terpenes such as d-limonene, N-methylformamide, stearic acid, polyethoxyethylenes, limonene oxides, hydrophobins, 3-methoxy-3-methyl-1-butanol, 3-methyl-1,3-butanediol, 1,3-propanediol, glycerol monopalmitate, dodecanoyl acetate, cetyl ethylhexanoate, stearyl ethylhexanoate, ethyl lactate, isobutyl lactate, triethyl acetyl citrate, phytantriol, salicylic acid and derivatives thereof, ascorbic acid and derivatives thereof, butylene glycol, thiazone, sodium dodecyl sulphate, nerolidol and 1,8-cineol, glycolipids, medium-chain triglycerides, branched-chain fatty alcohols such as octyldodecanol, taurine, phospholipids, 1,2-pentylene glycol and C8/C10-glycerol partial esters (esterification product of glycerol with a substoichiometric mixture comprising n-octanoic acid and n-decanoic acid), in particular C8/C10-glycerol partial esters as described for example in PCT/EP2011/056616. Particularly good values can be achieved using this additional auxiliary.

In a particularly preferred embodiment, the further components blended in process step B) are the partition modifier caprylic/capric triglyceride, which lead to a formulation with particularly strong penetration properties being obtained.

Preferably, in the process according to the invention, the provided composition and the further components are blended in a weight ratio of 1:5 to 1:5000, in particular from 1:8 to 1:12.

The examples below describe present invention by way of example without any intention of limiting the invention, the scope of application of which arises from the entire description and the claims, to the embodiments specified in the examples.

The following figures form part of the examples:

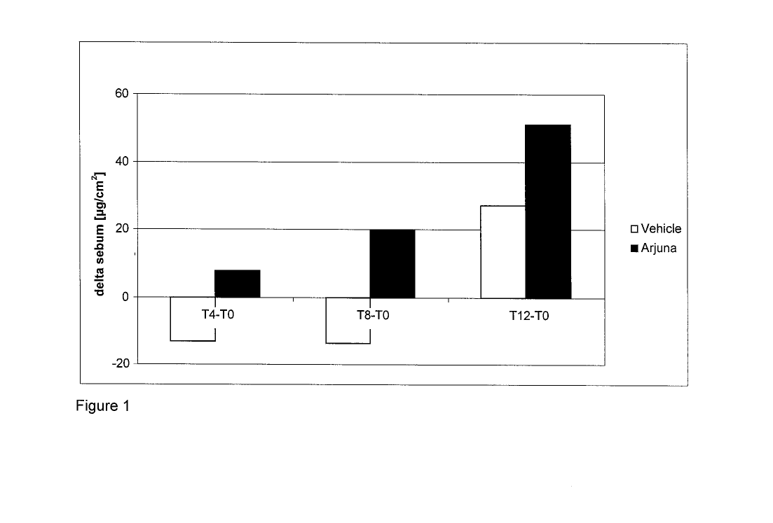

FIG. 1: Results of the sebum measurements

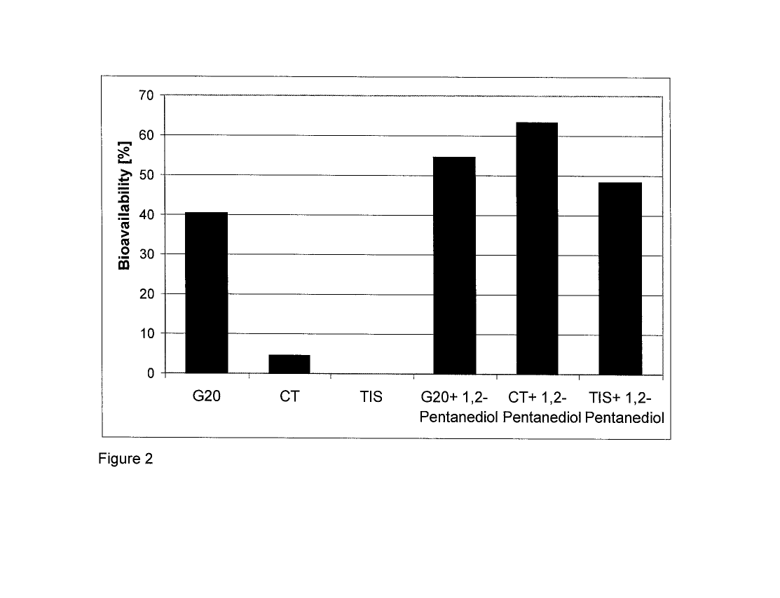

FIG. 2: Bioavailability as a function of solvent

### EXAMPLES

### Example 1

### Increasing the Sebum Production of Postmenopausal Skin by Means of Arjunolic Acid

The increase in sebum production of postmenopausal skin as a result of topical application of arjunolic acid in formulation was demonstrated in a human study.

The panel comprised 60 healthy female subjects of Caucasian origin up to seventy years old who were in their postmenopausal phase (average age 59.4 years). The formulation was applied on the face. Vehicle formulation (vehicle) and arjunolic acid formulation (arjuna) were applied by in each case 30 subjects. Application was twice daily, morning and evening, over a period of twelve weeks. Measurement points were made before the start of the application (T0), after four weeks (T4), after eight weeks (T8) and after twelve weeks (T12). The composition of the formulations is shown in the table below:

Test formulations. Data in percent by mass.

| | Raw material | INCI | Vehicle | Arjuna |
|---|---|---|---|---|
| A | TEGINACID ® C | Ceteareth-25 | 2.00 | 2.00 |
| | ABIL ® Care 85 | bis-PEG/PPG-16/16 | 1.00 | 1.00 |
| | | PEG/PPG16/16 | | |
| | | dimethicone; | | |
| | | caprylic/capric | | |
| | | triglyceride | | |
| | TEGO ® | Cetearyl Alcohol | 5.00 | 5.00 |
| | Alkanol 1618 | | | |
| | Stearin | Stearic acid (and) | 0.50 | 0.50 |
| | | palmitic acid | | |
| | TEGOSOFT ® APM | PPG-3 Myristyl ether | 4.83 | 4.83 |
| | TEGOSOFT ® G20 | Octyl dodecanol | 4.83 | 4.83 |
| | TEGOSOFT ® TN | C12-15 Alkyl benzoate | 4.83 | 4.83 |
| B | Water | Water | 71.8 | 71.6 |
| | Glycerol | Glycerol | 2.54 | 2.54 |
| C | TEGO ® | Carbomer | 0.15 | 0.15 |
| | Carbomer 134 | | | |
| | TEGOSOFT ® | | 0.60 | 0.60 |
| | APM/G20/TN 1:1:1 | | | |
| D | Arjunolic acid | Terminalia Arjuna | | 0.20 |
| | | Bark Extract | | |
| | 1,2-Pentanediol | Pentylene glycol | 1.80 | 1.80 |
| Z | Euxyl K220 | Methylisothiazolinone, | 0.12 | 0.12 |
| | | ethylhexylglycerin, water | | |
| | NaOH 10% | Sodium hydroxide | pH 5.5 | pH 5.5 |

To prepare the formulations, customary formulation processes were used. Skin sebum was measured using a Sebumeter SM 810 (Courage+Khazaka). The test was performed in a temperature- and humidity-controlled room (24±2° C., 50±10 relative humidity). The subjects were asked to not apply any product up to twelve hours before measurement and to wash their face three hours before measurement.

As FIG. 1 for the human in vivo study shows, a steady increase in sebum production was observed over a period of twelve weeks in the group of people who applied the arjunolic acid formulation. In the control group, who applied vehicle formulation, the value decreased within the first eight weeks following application. After twelve weeks an increase compared to the starting value was noted, although this was approximately half of that seen for the arjunolic acid formulation.

These results show that an increase in sebum production of the skin can be achieved on account of using arjunolic acid.

### Example 2

### Increasing the Bioavailability of Arjunolic Acid as a Result of Formulation with 1,2-pentanediol

The increase in the skin penetration of arjunolic acid from a cosmetic formulation as a result of adding 1,2-pentanediol was demonstrated in the course of a percutaneous absorption study on a pig skin model. In practice, the study was performed in accordance with a current protocol, as described for example in S. Richert, A. Schrader, K. Schrader, Int. J. Cosmet. Sci. 2003, 25, 5-13.

The investigated formulations are summarized in the table below.

| | | | | | G20 + 1,2- | CT + 1,2- | TIS + 1,2- |
|---|---|---|---|---|---|---|---|
| | Raw material | G20 | CT | TIS | pentanediol | pentanediol | pentanediol |
| A | TEGINACID ® C (Ceteareth-25) | 2.00 | 2.00 | 2.00 | 2.00 | 2.00 | 2.00 |
| | ABIL ® Care 85 (bis-PEG/PPG- | 1.00 | 1.00 | 1.00 | 1.00 | 1.00 | 1.00 |
| | 16/16 PEG/PPG16/16 | | | | | | |
| | dimethicone; caprylic/capric | | | | | | |
| | triglyceride) | | | | | | |
| | TEGO ® Alkanol 1618 (Cetearyl | 5.00 | 5.00 | 5.00 | 5.00 | 5.00 | 5.00 |
| | Alcohol) | | | | | | |
| | Stearic acid (and) palmitic acid | 0.50 | 0.50 | 0.50 | 0.50 | 0.50 | 0.50 |
| | TEGOSOFT ® G20 | 14.5 | | | 14.5 | | |
| | (octyldodecanol) | | | | | | |
| | TEGOSOFT ® CT (caprylic/capric | | 14.5 | | | 14.5 | |
| | triglycerides) | | | | | | |
| | TEGOSOFT ® TIS (triisostearin) | | | 14.5 | | | 14.5 |
| | Arjunolic acid | 0.25 | 0.25 | 0.25 | — | — | — |
| B | Water | 72.45 | 72.45 | 72.45 | 70.20 | 70.20 | 70.20 |
| | Glycerol | 3 | 3 | 3 | 3 | 3 | 3 |
| C | TEGO ® Carbomer 134 | 0.15 | 0.15 | 0.15 | 0.15 | 0.15 | 0.15 |
| | (Carbomer) | | | | | | |
| | TEGOSOFT ® Emollient (vide | 0.60 | 0.60 | 0.60 | 0.60 | 0.60 | 0.60 |
| | supra) | | | | | | |
| D | Arjunolic acid 10% in 1,2- | — | — | — | 2.50 | 2.50 | 2.50 |
| | pentanediol | | | | | | |
| Z | Euxyl K220 | 0.12 | 0.12 | 0.12 | 0.12 | 0.12 | 0.12 |
| | (methylisothiazolinone, | | | | | | |
| | ethylhexylglycerin, water) | | | | | | |
| | NaOH 10% (Sodium hydroxide) | 0.43 | 0.43 | 0.43 | 0.43 | 0.43 | 0.43 |

Test formulations. Data in percent by mass.

To prepare the formulations, customary formulation processes were used.

The results of FIG. 2 show that by using arjunolic acid in combination with 1,2-pentanediol in formulation it is possible to achieve an increase in bioavailability.

1. (canceled)

2. A method for increasing sebum production, said method comprising:
applying a cosmetic formulation comprising arjunolic acid to skin of a mammal.

3. The method according to claim 2, wherein said formulation further comprises asiatic acid.

4. The method according to claim 3, wherein said formulation further comprises terminalia arjuna bark extract.

5. The method according to claim 2, wherein said formulation further comprises 1,2-pentanediol.

6. The method according to claim 2 wherein said mammal is a post menopausal woman.

7. A composition comprising:
0.2% by weight to 20% by weight of arjunolic acid; and
80% by weight to 99.8% by weight of 1,2-pentanediol,
wherein the percentages by weight refer to the total composition.

8. The composition according to claim 7, further comprising asiatic acid.

9. The composition according to claim 8, further comprising terminalia arjuna bark extract.

10. A process for preparing a formulation, said process comprising:
providing a composition comprising 0.2% by weight to 20% by weight of arjunolic acid, and 80% by weight to 99.8% by weight of 1,2-pentanediol, wherein the percentages by weight refer to the total composition; and
blending said composition with at least one additional component of said formulation.
